(19) United States
(12) Patent Application Publication
(10) Pub. No.: US 2013/0057828 A1
(43) Pub. Date: Mar. 7, 2013

(54) HANDHELD PORTABLE FUNDUS IMAGING SYSTEM AND METHOD

(71) Applicant: Marc de Smet, Ottawa (CA)

(72) Inventor: Marc de Smet, Ottawa (CA)

(21) Appl. No.: 13/405,809

(22) Filed: Feb. 27, 2012

Related U.S. Application Data
(63) Continuation-in-part of application No. PCT/CA2009/001183, filed on Aug. 31, 2009.
(60) Provisional application No. 61/447,256, filed on Feb. 28, 2011.

(51) Int. Cl. A61B 3/15; G03B 15/02

(52) U.S. Cl. 351/207; 351/206; 351/246; 362/18

(57) ABSTRACT

A system and method for fundus imaging wherein selective illumination of a sector of the field of view of the fundus, using off-axis illumination, provides images of acceptable clarity, resolution, and size, with significantly reduced reflections, in a compact system. By rotating illumination around the optical axis, sectors of the fundus may be selectively and sequentially illuminated. An image of the entire field of view of the fundus is obtained by combining images, e.g. two or more half images, obtained within a single shutter exposure or capture period. An illumination system using LED light sources and a rotatable occluder provides for a lightweight, handheld and portable fundus imaging system. It may take the form of a fundus camera, a fundus imaging lens module for regular camera, or an adapter which couples to the standard lens of a camera to create a low cost fundus camera.

### CROSS-REFERENCE TO RELATED APPLICATIONS

This application is a continuation-in-part of PCT International patent application no. PCT/CA2009/001183, filed 31 Aug. 2009, designating the United States, entitled “Handheld portable fundus imaging system and method”. This application also claims priority from U.S. Provisional application No. 61/447,256 filed Feb. 28, 2011, entitled “Handheld portable fundus imaging system and method”. Both applications are incorporated herein by reference in their entirety.

### BACKGROUND OF THE INVENTION

1. Technical Field

This invention relates to fundus imaging systems and methods for retinal imaging and screening, and particularly to portable or mobile fundus cameras and camera adapters, suitable for handheld use, to enable remote deployment e.g. in the field, areas without clinic facilities, or for telemedicine.

2. Background Art

A fundus camera, or retinal camera, is a specialized low power microscope with an attached imaging device, designed to view and photograph or record images of parts of the interior surface of the eye called the fundus, which includes the retina, optic disc, macula and posterior pole.

Fundus cameras are used by optometrists, ophthalmologists, and other trained medical professionals for monitoring progression of a disease, diagnosis of a disease (e.g. combined with retinal angiography). They may also be used in screening programs, where the photos or images are analyzed at a later time and/or location. Fundus cameras have applications for both human and veterinary medicine.

Various medical conditions, such as diabetes, macular degeneration, or glaucoma, are widespread and an increasing health issue, in both developed and developing countries. Retinal imaging with a fundus camera provides a quick and effective way of screening for these conditions. It would be desirable to provide a lower cost system, suitable for use, in remote areas away from urban centres, or in developing countries which may not have ready access to large medical centres, or imaging centres. With the recent widespread availability of digital cameras at relatively low cost, it would also be desirable to be able to adapt commercially available cameras for such use to provide a compact low cost camera. However, available compact or portable systems do not address current requirements or do not provide sufficient image quality.

Fundus cameras are manufactured by companies including: Topcon, Carl Zeiss Meditec, Canon, Nidek and Kowa. Typically, fundus cameras are intended for fixed installation, e.g. in a hospital or clinic, and thus tend to be large, bulky and heavy. This is in part for ruggedness to protect optical components, and for stability, as required for conventional photographic imaging solutions in low light. These cameras also tend to be expensive, several costing more than $50,000. More recently, handheld or portable devices have been developed, for example, those manufactured by Kowa and Optimum Technologies.

For example, a Kowa RC-2 retinal camera is shown in FIG. 1, and although clearly intended to be portable, the handheld camera unit, having a pistol type grip, has a bulky separate power supply and connecting cables. While this camera is “portable”, it requires direct connection to a power outlet, an external light source, and uses conventional photographic film. It has been described by physicians as not handy to use, the field of view was limited, and reflections were a common problem. FIG. 2 shows a Kowa Genesis-D, which uses digital imaging, has an LCD monitor, and overcomes some of these problems. It provides for connection to a PC. However, it also has a rather heavy separate power supply/controller unit, which serves as a base support stand and a heavy cable tethering the unit to the base unit Kowa website http://www.kowa.co.jp/e-life/products/fc/index.htm; Handheld retinal camera http://www.kowa.co.jp/e-life/products/fc/genesis_d.htm and http://www.revophth.com/ProductGuide/pdf/pr0707_{—}21-28.pdf.

Nidek also manufactures a handheld fundus camera, the Nidek 200D (FIG. 3), which provides digital imaging, and has features such as auto focus and network connection. While relatively compact, it is stand mounted, rather than a handheld, portable unit. http://usa.nidek.com/products/afc.

Another retinal camera, called Retcam (FIG. 4) is described by Clarity Medical Systems, as having a particular application for screening of newborn babies, and is mounted on a mobile cart (http://www.claritymsi.com/retcamII.html). The features this camera has are many of the ones that a user wants in a portable unit. The wide field of view, up to 130°, fluorescein modality, and the ability to quickly check the quality of the image on a large screen, and video capability, make it a good design. However, the basic device remains large and manoeuvrability is limited. The imaging unit is compact, but the power supply control, display and analysis unit is cart mounted. The cart is suitable for mobile use in a doctor's office or hospital setting, but not suitable for portable or mobile field use. Its high cost has also been a severe limitation to its use in a number of hospitals and clinics.

Another ophthalmic handheld camera (FIG. 5) which is described as portable, is the Hawkeye slit lamp camera, which can be used with a 12V rechargeable power supply http://www.hawkeye-slitlamp.com/hawk-eye-caracteristiques-an-3.html. This is actually a table mounted unit, with a stand, LED source lighting, and a camera mounted on the top of the unit. To obtain images of the inside of the eye, an additional lens is required on the front surface of the camera unit. Due to the presence of reflections, obtaining images can be challenging particularly in uncooperative patients or patients unable to place their head in a chin rest.

Optimum Technologies describes a handheld retinal camera (see FIG. 6) http://www.optimum-tech.com/clientsandprojects/retinal_scope.cfm. Elements of this design are disclosed in U.S. Pat. No. 7,448,753 entitled “Portable Digital Medical Camera for Capturing Images of the Retina or the External Auditory Canal, and Methods of Use” to Chinnock. It is disclosed that this camera was designed to be lightweight and portable, and to be cordless; it is also disclosed that in order to keep the design compact, this camera provides a limited angle of view 30-50 degrees, which means the camera provides an image of a limited area of the fundus, and must be panned to provide examination of other areas. Such panning, unless done in a pre-determined sequence, can make interpretation of the findings difficult as landmarks within the eye are limited.

An article published in 2004 refers to the development of a low cost fundus camera for use with a standard Nikon camera developed by doctors Joseph M. Miller, James Schweigerling, and Robert W. Snyder at the University of Arizona. (http://www.opa.medicine.arizona.edu/ahsnews/may04/3profs.htm) and a design by these inventors is disclosed in U.S. Pat. No. 7,048,379 entitled “Imaging lens and illumination system” to Joseph M. Miller et al. To reduce glare, this system uses a ring illumination system, and a baffle arrangement to block illumination from a central region of an objective lens. Use of baffles significantly reduces the field of view.

Another example using ring illumination is described in U.S. Pat. No. 7,499,634, entitled “Ophthalmic camera, ophthalmic camera adaptor and methods for determining a haemoglobin and glucose level of a patient” to K. Yogesan, et al. (Lions Eye Institute in Australia). This system has a ring illumination arrangement, which is movable to ensure a circle of light is directed through the centre of an ophthalmic objective lens.

As mentioned in some of the above references, imaging of the eye presents particular challenges, that is, the eye itself is an optical system with its own lens and a highly reflective curved surface of the cornea, which tends to cause problems with reflections or glare when illuminating the eye. Also, the small size of the aperture presented by an undilated pupil, and the need for a wide angle of view to image the curved surface of the fundus, requires a short working distance. That is the camera must be placed close to, or in contact with, the eye. Moreover, the level of illumination that can enter the eye must be appropriately controlled. The optical system must be able to compensate for adaptation/accommodation by the eye when looking into the camera, and a typical range of refractive errors (myopia or hyperopia).

In compact handheld devices, limited space for optical components may necessitate changes to conventional optical systems in larger, bulkier conventional fundus cameras. In practice, for example, in attempting to shorten the optical path for compact systems, it is found that annular or ring illumination systems lead to problems with reflections, and poor image quality. Within the camera, reflections can be reduced to some extent by suitable optical coatings on lenses or reflectors, but these coatings tend to be a very expensive part of the optics, and add considerably to the cost of the optical system. Portable systems are also preferably low power, stand alone systems without the need for bulky power supplies, cables and connectors. However, in practice, existing portable systems have been found to have inconveniently short battery life.

Also, it would be desirable to have a low cost unit, which is durable, and easy to use, for deployment in rural areas of developing countries where regular medical facilities or specially trained personnel may be limited. Thus, there is a need for an improved, low cost, handheld fundus camera system for retinal imaging, which would be suitable for telemedicine in remote areas, and in the field, where equipment must be taken to the patient.

### SUMMARY OF INVENTION

An object of the present invention is to mitigate the above-mentioned limitations or deficiencies of known fundus cameras, or at least provide an alternative.

One aspect of the invention provides a fundus imaging system comprising first and second optical elements defining an optical axis for alignment to an eye to be imaged; the first optical element comprising an objective lens for focusing an intermediate image of the fundus of the eye at an intermediate focal plane; the second optical element for coupling the intermediate image for imaging the fundus in an image plane of an image sensor; illumination means comprising an off-axis light source and an occluder for selectively illuminating a sector of the fundus within a field of view; and control means for sequentially positioning the illumination means for illumination of a first sector of the field of view of the fundus, actuating the light source and capturing an image of the first sector, and then, positioning the illumination means for illumination of a second sector of the field of view of the fundus, actuating the light source and capturing an image of the second sector.

A second aspect of the invention provides a method for fundus imaging comprising selectively illuminating a first sector of the field of view of the fundus, and capturing an image of the first sector, selectively illuminating a second sector of the field of view of the fundus and capturing an image of the second sector, and combining images of first and second sectors.

Another aspect of the invention provides a method for fundus imaging in a fundus imaging system having an optical axis defined by optical elements of the imaging system comprising an ophthalmic objective lens for optical alignment with an eye to be imaged, the method comprising: providing a light source at a point position on a ring around the optical axis, and located relative to the intermediate focal plane of the aspherical lens for focusing an off-axis spot of light in the pupillary plane of the eye to be imaged; from a first light source position selectively illuminating a first sector of a field of view of the fundus and capturing an image from the illuminated first sector of the fundus; from a second light source position selectively illuminating a second sector of the field of view of the fundus and capturing an image from the illuminated second sector of the fundus; and combining images from at least first and second sectors to provide an image of at least part of the field of view of the fundus.

Selectively illuminating a sector of the fundus may comprise placing an occluder, such as a semicircular occluder element, near an intermediate focus point of the aspheric lens to occlude light from the first light source position to other sectors during illumination of the first sector of the fundus, e.g. half the fundus, and rotating the occluder to occlude light from the second light source position to other sectors during illumination of the second sector the fundus, i.e. the other half of the fundus. Sequential first and second half images of the fundus may be combined to provide an image of the field of view of the fundus. Optionally, a second occluder element may be placed closer to or coplanar with the light source to block unwanted reflections from a corneal surface of the eye.

Other aspects of the present invention provide a fundus camera, a camera adapter for fundus imaging, and an illumination system for fundus imaging, which provide for selectively illuminating a sector of the field of view of the fundus, and capturing an image. The system comprises control means for rotating illumination around the optical axis so that other sectors of the field of view of fundus may be sequentially imaged in the same way, and images combined to provide an image of the entire field of view of the fundus, preferably within a single shutter exposure interval or image capture period.

Preferably, illumination for fundus imaging is provided by an off-axis light source, i.e. radially spaced from the optical axis. Since the illumination path is off centre from the optical axis, reflections are separated from the optical path of the image. For example, by using an occluder to block, e.g., one half of the field, while illuminating the other half of the field of view of the fundus, an image of that half of the fundus may be obtained with significantly reduced reflections. An image of the entire field of view of the fundus may be constructed by combining two or more half images. Preferably, an illumination system is provided comprising a rotatable occluder, and a set of a plurality of light sources, e.g. four LEDs arranged in a ring, for illuminating sequentially half of the fundus from four positions. By appropriately synchronizing rotation of the occluder and flash of the four light sources during a single shutter exposure, an image of the entire field of view of the fundus may be generated by combining sequential images.

In another preferred arrangement, a point light source is provided that is spaced from and rotatable around the optical axis, and the occluder comprises first and second occluder elements. The first occluder element is provided as described above to allow illumination of a selected sector of the field of view. A similar second occluder element is placed close to or substantially in the plane of the light source to block unwanted reflections from a corneal surface of the eye. The second occluder element is aligned with respect to the first occluder element and rotatable synchronously with the first occluder. More preferably, the point light source is supported on the second occluder element and thus the light source rotates synchronously with the occluder elements to illuminate sequentially sectors of the field of view of the fundus. For example, the light source may be rotated to first, second, third and fourth positions as described in the previous embodiment to illuminate four successive sectors, which may each be halves of the total view of the fundus. However, it will be appreciated, this arrangement beneficially provides for the illumination means comprising the first and second occluder elements and the light source to be continuously rotated during an image capture period, while collecting image data from successive sectors of the field of view of the fundus to construct an image of the entire field of view.

Conveniently, a fundus imaging lens module is provided which may be used with a standard exchangeable lens camera, e.g., a D-SLR, using a conventional lens mount, power and control connections.

Beneficially, the fundus camera adapter may be attached to a standard camera to provide fundus imaging capabilities using almost any conventional, low cost, off the shelf camera, e.g., having a standard camera lens. Advantageously, the adapter comprises a housing/body, which provides a handle for handheld use, with shutter control and power connections, and/or illuminator controls, and a power supply.

Preferably, the optical imaging system used an aspheric ophthalmic objective lens that provides for a wide-angle field of view, up to 120 to 130 degrees, and uses a minimal number of low cost optical elements, and a solid-state LED illuminator. Preferred embodiments are sufficiently rugged for field use, and may be manufactured with low cost. Advantageously, a camera is provided which is a single lightweight unit, which may be handheld.

In preferred embodiments, a method and system for fundus imaging is provided wherein illumination is provided by a light source radially spaced from the optical axis and located relative to the intermediate focus plane of an aspherical ophthalmic lens so as to focus an off-axis point of light in the pupillary plane of the eye to be imaged. By providing an off-axis illumination path at an appropriate illumination angle, reflections are separated from the optical path of the image. For example, by using a point light source and an occluder and selectively illuminating only part, i.e., a sector, or half of the fundus, an image of that part may be obtained with significantly reduced reflections. By rotating the illumination around the optical axis relative to the fundus, other sectors of the field of view of the fundus may be illuminated sequentially, and an image of the entire field of view of the fundus may be obtained by combining sequential images, preferably within a single exposure period during which the light source is flashed at two or more positions. For example, after positioning the illuminator to illuminate half the fundus and recording a first image during a first flash, rotating the illuminator 180 degrees and recording an image of a second half of the fundus during a second flash, to provide two half images that are combined in a single shutter exposure period to provide an image of the entire field of view of the fundus. This method of illumination provides images of acceptable clarity, resolution, and size, with significantly reduced reflections, in a compact system. In particular, this illumination system provides for a lightweight, handheld and portable fundus camera (retinal imaging) system, which may take the form of a fundus camera, a lens assembly for adapting a regular camera for use as a fundus camera, or an adapter, which attaches to a regular lens of a camera to create a low cost fundus camera.

With suitable illumination sources and/or filters for different coloured illumination, various imaging modalities may be provided to allow for angiography, oxymetry, red free imaging, or autofluorescence, for example. With suitable additional lenses, such a camera may also be adapted for other applications, such as biometric imaging of the retina or iris, for use in other external medical imaging applications in the field.

While embodiments of the system and method are particularly directed to ophthalmic applications, another aspect of the invention provides an illumination system for wide angle imaging through an orifice using a similar off-axis illumination source and rotatable occluder to reduce reflections, e.g. for endoscopy, other medical, non-medical or diagnostic applications.

Other aspects and features of the present invention will become apparent to those ordinarily skilled in the art upon review of the following description of specific embodiments of the invention in conjunction with the accompanying figures.

Embodiments of the invention will now be described by way of example only with reference to the accompanying drawings.

### BRIEF DESCRIPTION OF DRAWINGS

In the drawings, identical or corresponding elements in the different figures have the same reference numeral [or corresponding elements of different embodiments have a reference numeral incremented by 100, i.e. are numbered 120, 220, 320, etc.].

FIGS. 1 to 6 show photographs of examples of known commercially available fundus camera systems;

FIG. 7 shows a schematic representation of key elements of an eye;

FIG. 8 shows a schematic representation of elements of an optical system for a fundus imaging system using a classical fundus illumination system and an LED ring light;

FIG. 9 shows a schematic representation of elements of an optical system for a fundus imaging system using LED ring illumination placed close to the intermediate focus plane of an aspheric lens;

FIG. 10 shows a schematic representation of elements of an optical system for a fundus imaging system using an illumination system and method according to a first embodiment of the present invention;

FIG. 11 shows a corresponding ray trace diagram, for part of the optical system of FIG. 10 for illumination of half of the fundus;

FIG. 12 shows a schematic representation of an assembly of a camera and elements of the optical system for a fundus imaging system according to a second embodiment;

FIG. 13 shows a schematic diagram of the LED illumination and occluder of the fundus imaging system shown in FIG. 12, and FIGS. 13A, 13B, 13C and 13D shows positioning of the occluder and light source activated for sequential illumination of sectors of the fundus; FIGS. 13E and 13F shows alternative occluder configurations;

FIG. 14 shows a photographic sample image of half of field of view (130 degrees) the fundus of a human eye generated by the system of FIG. 12, using a semicircular occluder (visible in the lower portion of the image) for illumination of half the field of view;

FIG. 15 shows a perspective view of the assembly similar to that in FIG. 12, showing the fundus imaging system adapter assembled to a conventional camera;

FIGS. 16 and 17 show two perspective views of the assembly of FIG. 15, with the cover removed to show internal optical elements and other components;

FIG. 18 shows schematically a fundus imaging system according to a third embodiment comprising a lens module;

FIG. 19 shows schematically a fundus imaging system according to a fourth embodiment comprising a camera;

FIG. 20 shows an exemplary photographic fundus image of a 130-degree field of view of a pig eye obtained with the fundus imaging system shown in FIG. 12;

FIG. 21 shows a schematic view a fundus imaging system according to a fifth embodiment;

FIG. 22 shows a perspective view of part of the system shown in FIG. 21 showing detail of the rotatable illuminator; and

FIG. 23 shows an exemplary photographic image of a 130 degree field of view of a pig eye generated with the system shown in FIGS. 21 and 22.

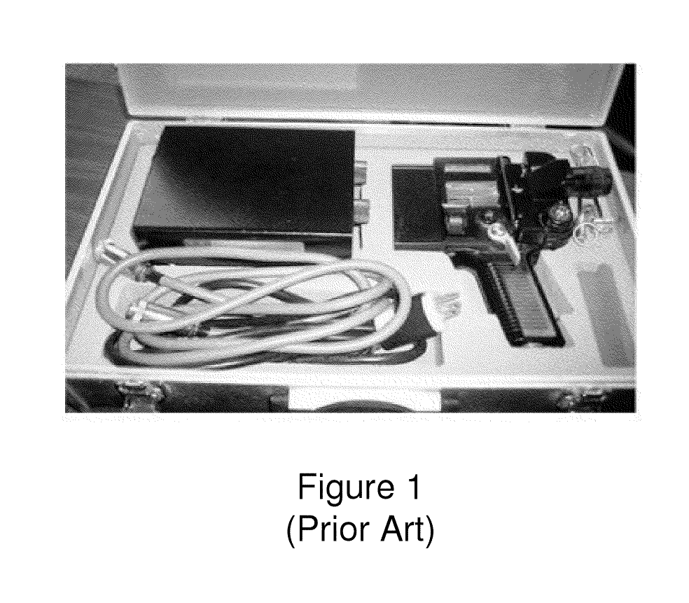

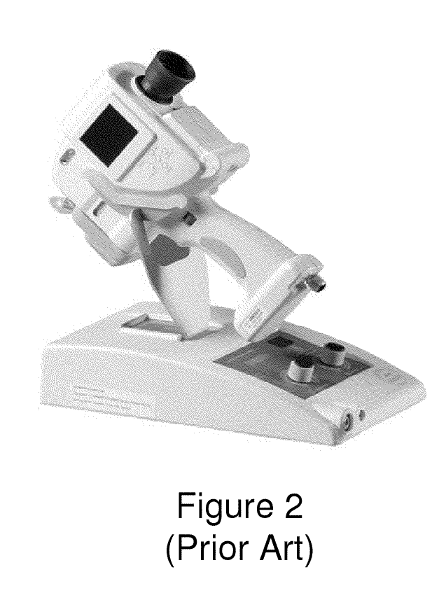

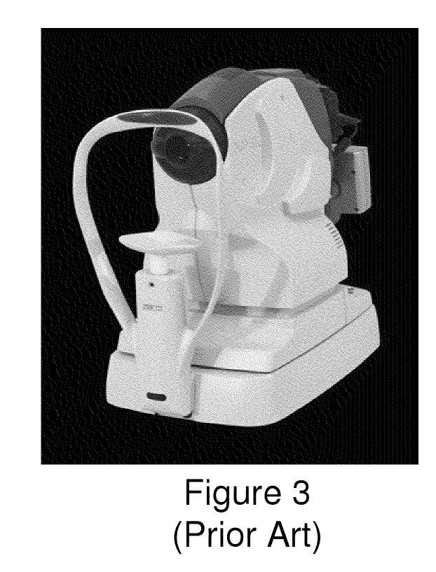

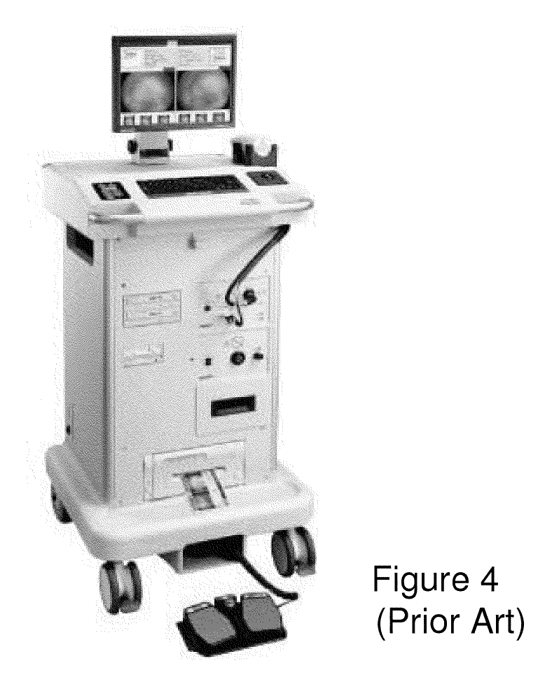

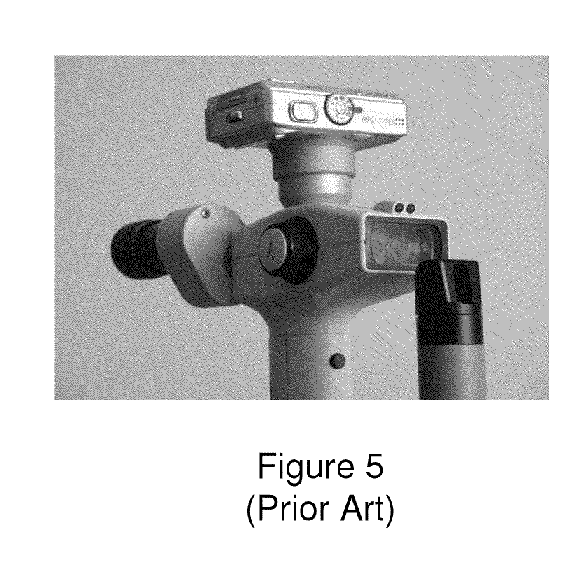

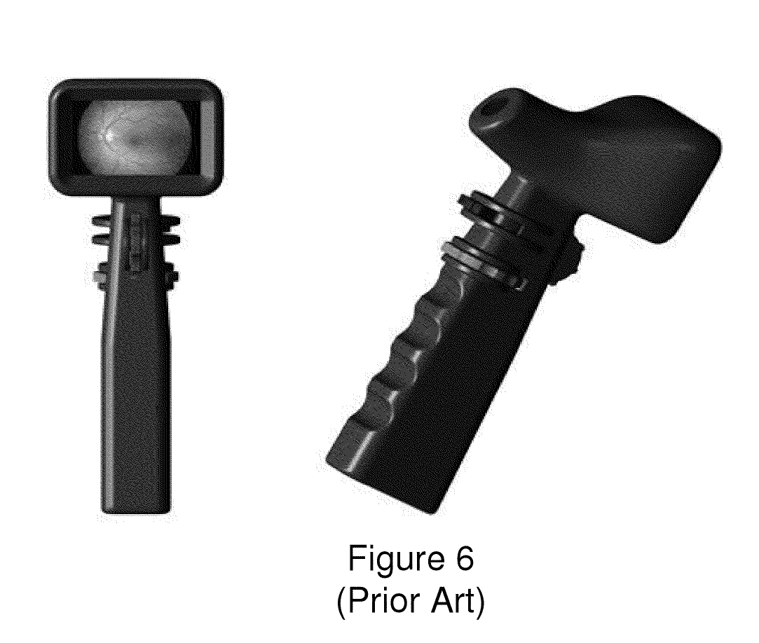

### DESCRIPTION OF PREFERRED EMBODIMENTS

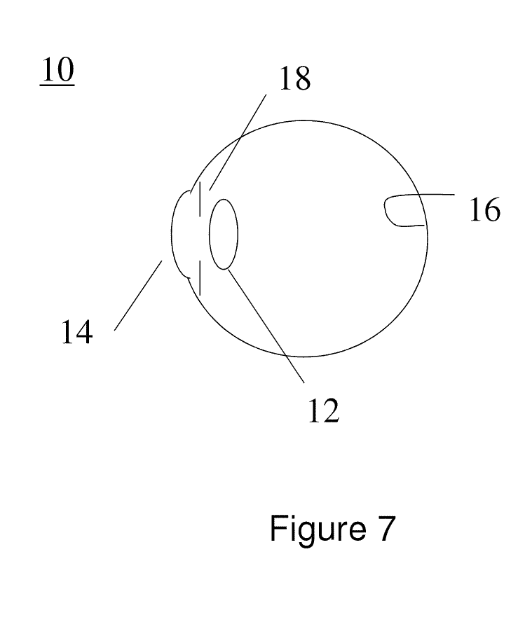

As shown schematically in FIG. 7, an eye 10 is a near spherical optical system itself, comprising the lens 12, the curved surface of the cornea 14, and the fundus 16, which is the curved structure at the back of the eye, comprising the retina, optic disc, macula and posterior pole. This structure presents some challenges in providing even illumination, through the lens 12 into the eye, and obtaining images over a wide field of view, preferably from at least 50 degrees up to 130 degrees. The pupil 18, which defines the pupillary plane, acts as a variable aperture into the eye, which may dilate or contract. To obtain good quality images, over a wide field of view, the objective lens of a fundus imaging system, which is typically a type of wide-angle aspheric lens, must be placed in close proximity with, or in contact with, the front surface or cornea 14 of the eye. That is, the working distance may be a few millimeters to a few centimeters. Of course, for human or veterinary ophthalmology, the subject may move or react to examination. The eye may adapt or accommodate to external illumination and optics during imaging and examination, resulting in some additional challenges relative to imaging of other anatomical structures. Furthermore, the surface of the cornea is highly reflective and the spherical structure of the eye, and the lens, may contribute to reflections within a camera system, which interfere with image quality.

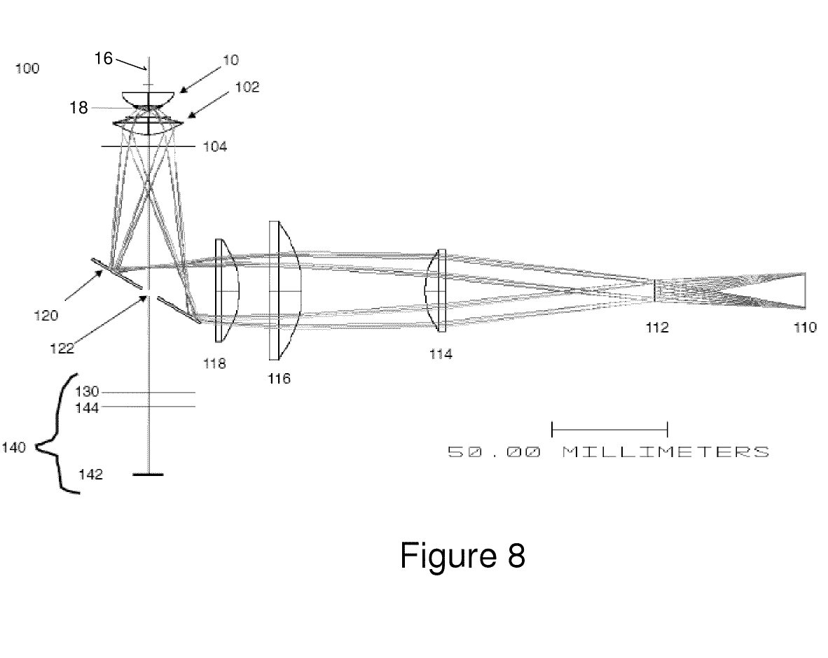

The optical elements of an optical system for a fundus camera 100 using a classical fundus illumination system are shown for reference in FIG. 8. This arrangement for illumination is that typically used in commercially available fundus cameras and imaging systems, in which light is sent to the eye via a partially transparent reflector, or a minor having a central aperture. As shown in FIG. 8, the system 100 comprises an aspherical objective lens 102. The illumination system comprises a light source, e.g. a solid state LED ring light source 110, and achromatic lens 112, and three other singlet lenses 114, 116 and 118 for imaging a ring of light from the light source 110 on a partial reflector or mirror 120 having a central aperture 122 and directing light from the illuminator 110 along the optical path of the imaging system, through the aspheric lens 102 and into the eye 10. The aspheric lens 102 collects light reflected from the eye 10, focusing an image of the fundus 16 in the intermediate image plane 104 of the aspheric lens 102. Illumination from light source 110 is provided in such a way as to project a ring of light to the outer area of the pupil 18 of the eye 10. The inner area of the optical system is used for the light path for collection of light reflected from the curved surface of the fundus 16 inside the eye 10 to a camera or a digital imaging system 140. Although a conventional photographic film camera may be used, the camera 140 is preferably a digital camera and comprises an imaging device/sensor, which is CMOS/CCD chip 142 and camera objective lens 144, which is coupled to the other optics via an achromatic lens 130.

The aspherical lens 102 is an ophthalmic wide-angle lens, similar to a slit lamp lens, for collecting light from the fundus 16 of the subject eye 10, and generating an intermediate image in plane 104. The aspheric lens 102 determines the distance of the minor, which corresponds to the imaging distance for the pupil through the aspheric lens 102. In the example shown in FIG. 8, this plane is about 105 mm from the intermediate focusing plane of the aspheric lens 102. The diameter of the light bundle collected from the eye in the direction of the imaging sensor 142 determines and must fall within the diameter of the aperture through the mirror. The image of the LED ring of illumination must fall outside the area of the aperture through the mirror. In practice, this requires that relatively large diameters are used for the lenses 114, 116, and 118 in the illumination path. For a compact system, the distance between the aspheric lens and the image plane of the sensor is preferably about 100 mm to 150 mm. As illustrated in FIG. 8, to provide a relatively compact system, an illumination system is provided comprising an achromatic lens 112 of focal length 65 mm and three singlet lenses 114, 116 and 118 of focal lengths 150 mm, 254 mm and 185 mm, respectively. In the illumination path, a black dot in the middle of the achromatic lens 112 is provided to ensure no light is reflected directly along the optical axis from the cornea of the eye, or from one of the surfaces of the aspherical lens in the direction of the image sensor 142. In this system, a limiting factor is the projection of the LED ring 110 at the pupil is the focusing by the aspheric lens 102 of the image at the plane of the minor 120.

Images taken of a human eye, with a dilated pupil, using the set up illustrated in FIG. 8 showed a significant number of reflections. This system is sensitive to small aberrations in the position of the eye, and the curvature of the eye in relation to the position of the optical components, and careful alignment is needed to reduce reflections. To reduce the size of the illumination system, the optical length may be folded by adding an additional lens, but relatively large diameter lenses are required (about 70 mm). Consequently, this type of arrangement has a number of limitations when trying to design a compact system, and reflections limit image quality.

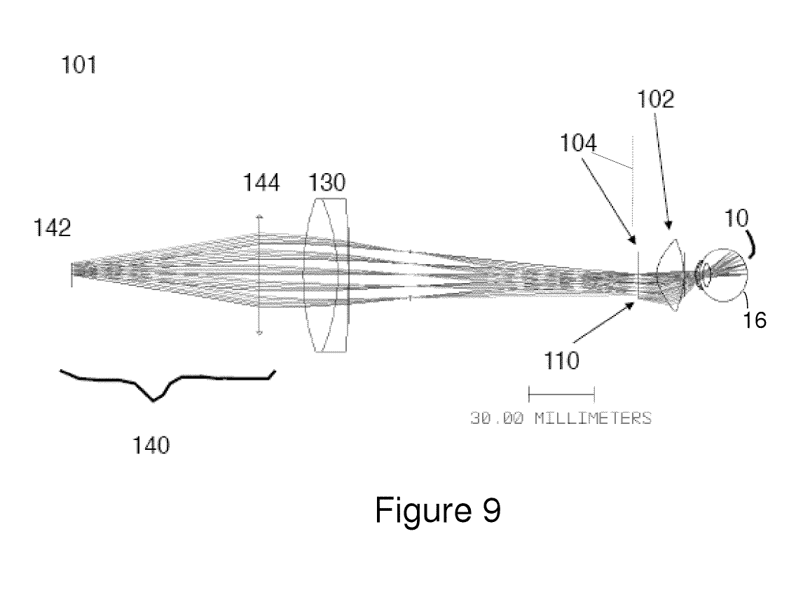

In an attempt to reduce reflections, an alternative illumination system was tested. This system 101, shown schematically in FIG. 9, comprises a similar aspheric objective lens 102, a LED ring light source 110, an achromatic lens 130 coupled to a digital camera 140 comprising an objective lens 144 and imaging sensor 142. However, the LED ring illuminator 110 is placed at or near the intermediate focus plane 104 of the aspheric ophthalmic lens 102. Thus, an illuminated ring is generated on the fundus just outside the imaging area of the camera. Light from the illuminator passes through the cornea 14 and lens 12 of the eye, and is reflected from the curved surface of the fundus 16. The aspheric lens 102 is used to image the fundus at an intermediate focus plane 104, and the achromatic lens 130 coupled to the camera objective 144 focuses an image of the fundus in the plane of the image sensor 142 of the camera 140. However, it was found that in practice this system provided an insufficient level of retinal illumination to effectively image the fundus. To overcome these problems an alternative system was sought.

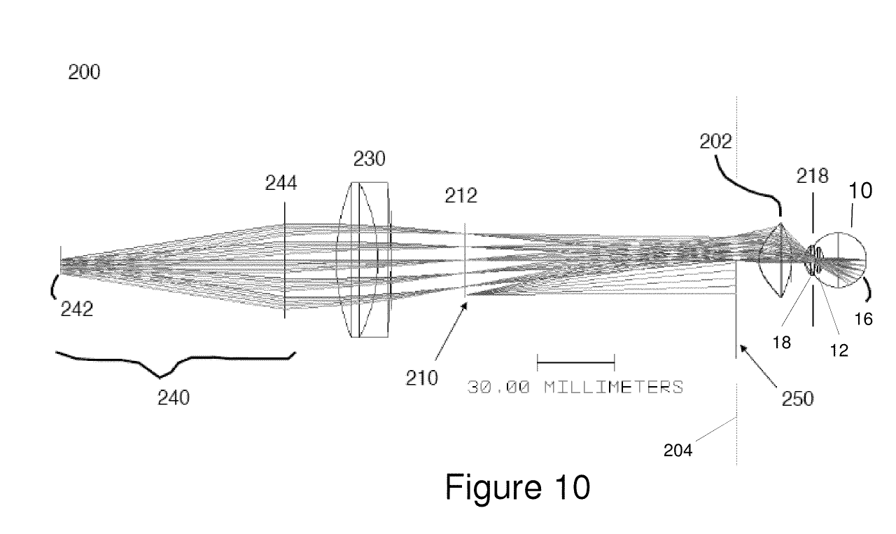

Optical elements of a fundus imaging system 200 according to a first embodiment of the present invention are shown schematically in FIG. 10. This system 200 comprises an aspheric ophthalmic lens 202 for focusing light from an LED illuminator 210 into the eye 10 through the pupil 18 and lens 12 of the eye 10, to illuminate the fundus 16. The double aspheric lens 202 collects light reflected from the fundus 16, focusing it at the intermediate focus plane 204 of the aspheric lens 202, and light is directed through an achromatic lens 230 into the objective lens 244 of a camera imaging system 240, to image the fundus in the plane of the image sensor 242 of the camera. The LED illuminator 210 is placed at a position 212 along the optical axis of the system so as to produce, through the aspheric lens 202, a sharply focused image of the illumination source at the pupillary plane 218. An opaque occluder 250 is provided so as to occlude or block illumination to part of the field of view and selectively illuminate only part of the field of view of the fundus, in this example, a sector comprising half the field of view, using a single LED light source 210. As shown in FIG. 10, this position of the LED light source 210 corresponds to a distance of about 105 mm from the intermediate focus plane 204. The LED light source lies at a radius of about 14-17 mm outside the optical axis of the imaging system. This off-axis positioning of the light source 210 is determined to reduce optical reflections while maintaining a suitable pupil size. That is, positioning the LED light sources radially closer to the optical axis, providing a smaller illumination angle, would risk optical reflections, while placing the LED light sources at a larger radial distance from the optical axis, at a larger angle, from the optical axis would require a larger pupil size, or risk that the field would not be fully illuminated through the pupil.

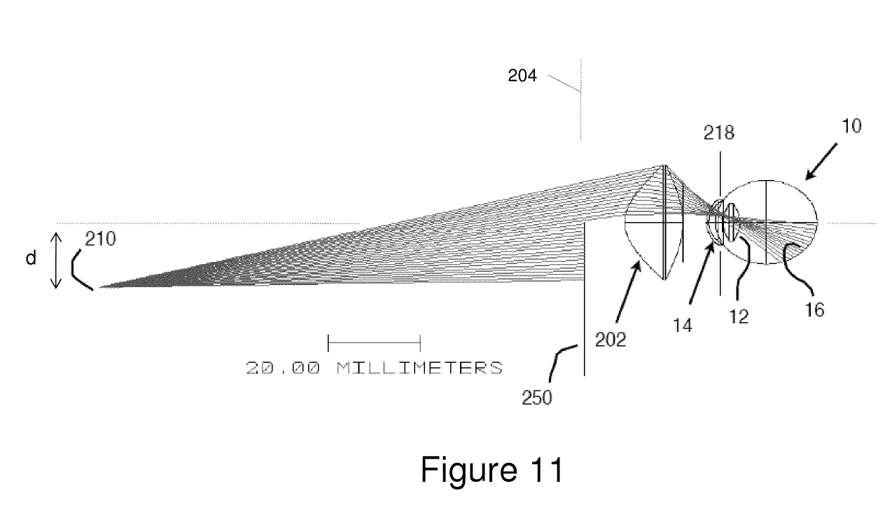

FIG. 11 shows a corresponding ray trace diagram for illumination of half of the fundus 56 by a single LED 210, shown as a point light source, and positioned as described above, radially spaced a distance d relative to the optical axis of the system. FIG. 11 also shows the position of occluder 250, which is semicircular, and aspheric lens 202 providing for illumination of part of the fundus 16, in this case a sector comprising one half of the field of view of the fundus 16. Illumination to the other half of field of view of the fundus is blocked by the semicircular occluder 250, which is positioned on the same side of the optical axis as the light source 210. Thus, an image may be recorded of half the fundus with the occluder blocking the lower half of the image, as represented in FIGS. 10 and 11. Using this illumination arrangement, a spot of light is focused in the pupillary plane 218, radially spaced from the centre of the pupil, so that light enters the eye off-axis, and illuminates half of the fundus. That is light from the point light source position on one side of the optical axis is directed across the optical axis and illuminates a sector, in this case half of the fundus on the opposite side of the optical axis, while direct illumination on the same side of the optical axis as the light source is blocked by the occluder 250. This arrangement significantly reduces glare and reflections from the corneal surface.

To obtain an image of the other half of the fundus, the occluder 250 and illumination source 210 are rotated about the optical axis relative to the fundus, to provide illumination of the other half of the field of view of the fundus and a second image recorded. Illumination of half the fundus at a time was found to provide superior quality images with significantly reduced reflections. By recording and combining two images of respective opposite halves of the fundus, an image of the entire field of view of the fundus may be generated. This approach allows for high quality images of the fundus to be obtained over a wide field of view of up to 130 degrees.

Thus, a method is provided in which selective illumination of a part of the fundus, e.g. a sector comprising half the field of view of the fundus in a first exposure is used to generate an image of the corresponding half of the fundus. Then illumination of the second half of the field of view of fundus is used to generate a second image of the other half of the fundus. Preferably, the two exposures are then combined to provide an image of the required field of view of the entire fundus. In this way, a wide field of view of the fundus may be imaged in two halves, with a wide field of view and with reduced reflections relative to full illumination of the fundus.

When using a digital camera, two half fundus images may be combined electronically. Alternatively, as will now be described, a system may be provided to allow synchronized flash illumination of two or more half fundus images during a single shutter opening period (or image capture period) to enable an image of the full field of view of the fundus to be obtained in a single exposure with a reasonably fast shutter speed.

### Camera Adapter

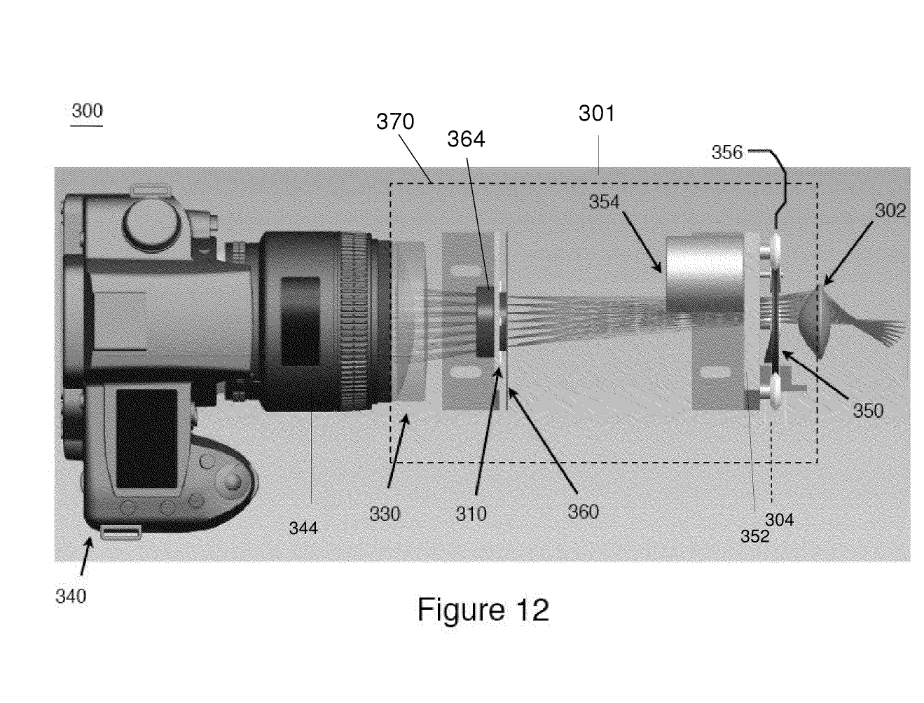

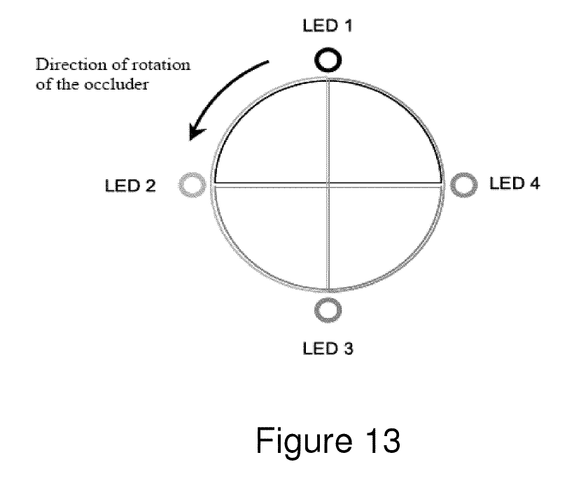

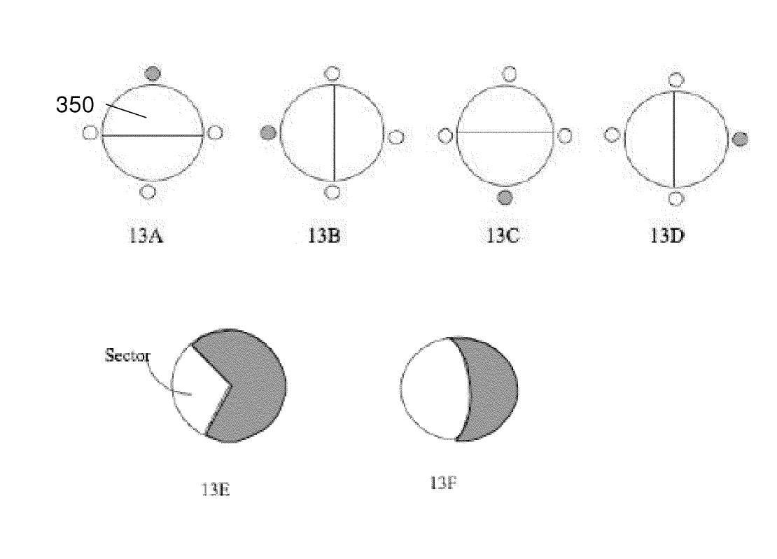

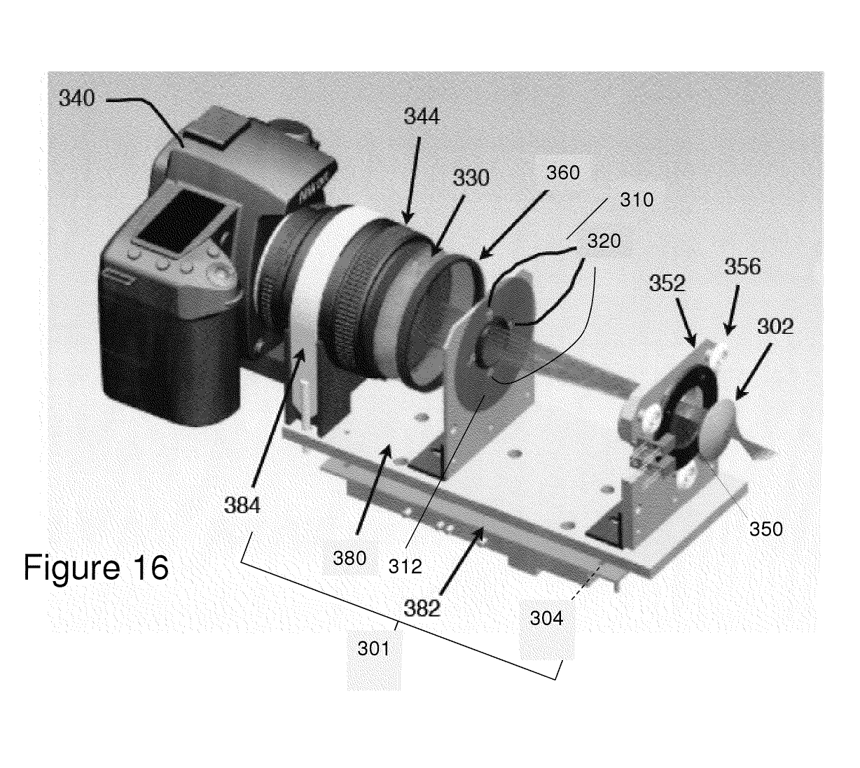

In a practical implementation, an optical system 300 for imaging of interior parts of an eye 10, according to a second embodiment of the present invention, is illustrated schematically in FIG. 12. The system 300 comprises an assembly of a fundus imaging system adapter 301 and a conventional digital camera 340 having an off the shelf camera objective lens 344. The adapter 301 is enclosed in a housing 370 (not shown in FIG. 12) and is coupled to the camera objective via an achromatic lens 330. The other optical elements of the fundus imaging camera adapter comprise an aspherical ophthalmic lens 302, an LED illuminator 310, and a polarizer 360. A semicircular occluder 350 is mounted on a motorized mount 352, including a small motor 354 and drive system 356 to allow the semicircular occluder 350 to be rotated around the optical axis of the system. Referring to FIGS. 13 and 16, which show the arrangement of the LED illumination system 310, four individual light emitting diodes (LED) LED1, LED2, LED3 and LED4 (320a, 320b, 320c, 320d) are arranged in a ring on an annular support 312, placed concentrically around the optical axis. The four LEDs (320a, 320b, 320c, and 320d) are illuminated sequentially while the semicircular occluder is correspondingly rotated around the optical axis to each of four positions as shown in FIGS. 13A, 13B, 13C and 13D, to allow for sequential illumination of half of the field of view of the fundus by each of the four LEDs 320. Using this configuration, an image of the entire field of view fundus may be constructed using four half images collected during one full rotation of the occluder.

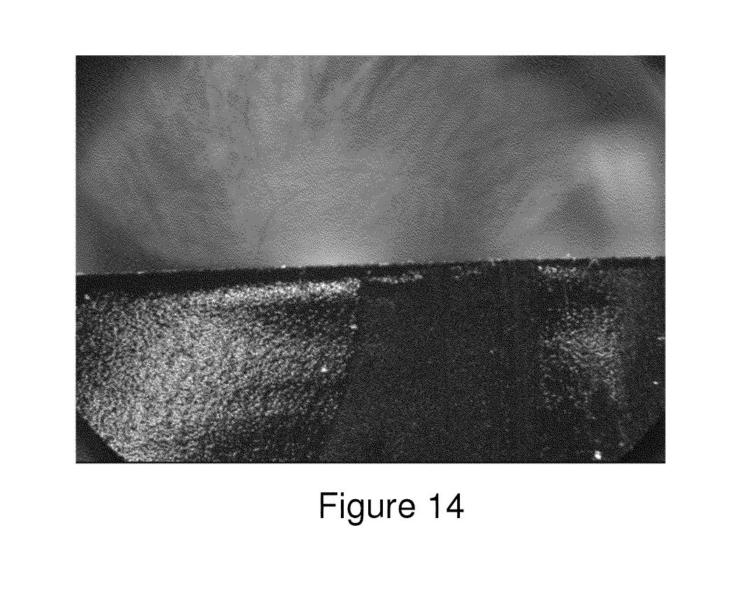

The corresponding ray trace diagram for illumination by each individual LED 320 is similar to that shown in FIG. 11. As shown in FIG. 16, the LEDs 320 lie on a radius of about 14-17 mm outside the optical axis at a distance of about 105 mm from the intermediate focal plane of the aspheric lens 302. At the intermediate focal plane 304, the semicircular occluder 350 occupies half the field on the same side of the optical axis as the LED which is to be illuminated. A sample image of one half view of half the fundus is shown in the upper half FIG. 14, while the occluder 350 occupies the lower half of the image. The occluder is preferably as optically dark as possible, e.g. a non-reflective black plastic and preferably the occluder is positioned/oriented at an angle of at least 10 degrees off-axis, to reduce back reflections directly along the optical axis. In the plane of the LEDs, a diaphragm 364 is placed in the light path of the camera to the eye (FIG. 12). This diaphragm 364 is used in imaging the eye, to reduce reflections in the direction of the camera. In this embodiment the maximal aperture is set at 22 mm. The camera objective 344 must then have a minimum opening corresponding to an F-number of 2.0. The minimal opening of the diaphragm is 10 mm, and that of the camera objective is about 3.4 mm. To further reduce reflections, a polarizer 360 is set in front of the LEDs, the camera and the achromatic lens. Direct reflections may therefore be further reduced. For example an Edmund Optics 45-668 polarizer provides good contrast with crossed polarizers providing a transmission of about 0.04%. Since the mounting or housing (not shown in FIG. 12) surrounding the aspheric lens 302 also contributes further reflections, any light from the LED at the edge of the first lens surface reflects on the housing of the lens and will ultimately head in the direction of the camera. Thus, the housing surrounding the aspheric lens should also be made as optically opaque and non-reflective as possible. The remainder of the internal surface of the housing is also preferably optically black, and may provide a tubular covering over optical elements as is conventional for optical equipment. Preferably a target image, e.g., a red target light spot is provided for the subject to focus on during imaging. The target may be movable so that the patient can be asked to orient the eye for imaging further into the eye.

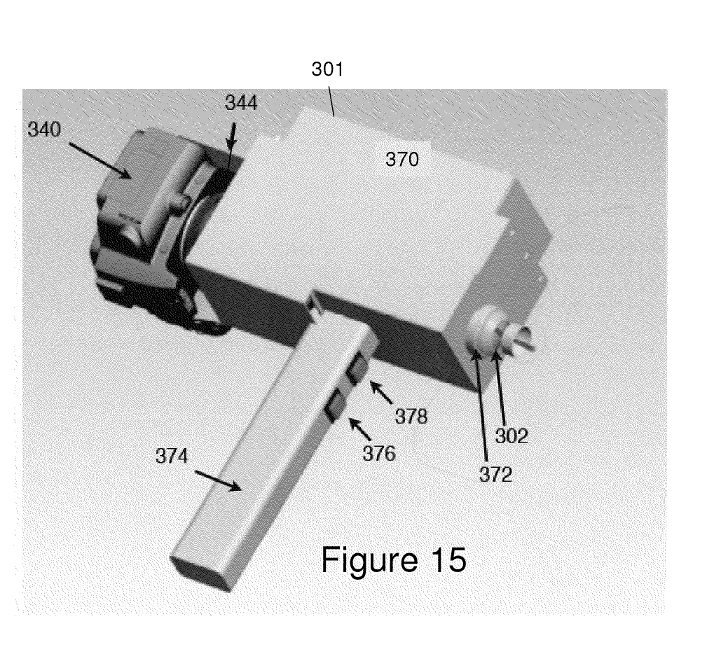

FIGS. 15, 16 and 17 show other views of a prototype system similar to that shown in FIG. 12. As shown in FIG. 15, the optical and electronic components of the fundus imaging adapter 301 are enclosed within a housing 370, which is coupled to the objective lens 344 of the camera 340. The lens mount 372 for the aspheric ophthalmic lens 302 provides for fine focus adjustment as will be described below. Controls, e.g., illumination and shutter control buttons 376 and 378, are provided on a handle portion 374 of the housing 370. Conveniently, the handle 374 also accommodates batteries or a battery pack for powering the adapter. FIG. 16 shows a view with a cover of the housing 370 removed, the optical elements are mounted on a support surface 380 which also supports a circuit board 382 accommodating the electronics and control system. The housing 370 of the adapter 301 is coupled to the objective lens 344, for example, by a lens mount coupling or other suitable fastening means e.g., a clamp 384, that secures and optically aligns the adapter 301 to the camera objective 344 and provides appropriate power and control couplings to the camera, as required.

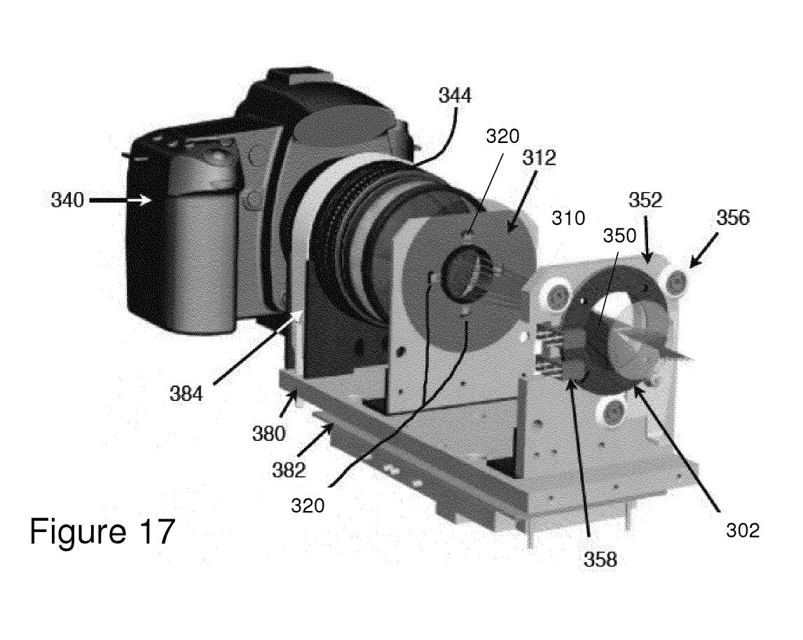

FIG. 17 shows another view of the camera adapter 301 showing the arrangement of the LED illuminator 310, comprising four LEDs 320 arranged at 90 degrees around the annular support 312. Also shown, is the support 352 for the rotatable semicircular occluder 350, drive means 356, and power connections 358 for the motor drive.

The device housing 370 is preferably lightweight and designed to be compact (i.e. <100 mm-150 mm long) to facilitate handheld use, preferably with an ergonomically designed handle and controls.

In operation, to generate an image of the entire field of view of the fundus, which may be up to 130 degrees, an image is constructed from four half images. As the occluder 350 is rotated sequentially to the first, second, third and fourth positions, as shown schematically in FIGS. 13A, 13B, 13C and 13D, the individual LEDs 320 are turned on, i.e. flashed, in sequence as the occluder is rotated to the corresponding position, so as to selectively illuminate the respective half of the field of view of the fundus. An appropriate shutter speed is selected for the camera, and the speed of rotation of the occluder is synchronized to the timing of sequential illumination of each of the LEDs, the respective LED being flashed when the occluder reaches the corresponding position to provide illumination of half the field of view, to enable four flash images, or exposures, to be collected during the shutter opening period. Control circuitry provides for the LEDs to be flashed on and off synchronously with rotation of the occluder to the appropriate position, i.e., in a strobe like mode. As the occluder is rotated, each LED is individually flashed synchronously at the appropriate time to illuminate the corresponding half of the field of view. Thus, an image is recorded from the sum of the image data resulting in four separate flash exposures made during the shutter open time of the camera.

For example, the occluder may be rotated at 1200 rpm (20 rotations per second). This implies in 50 milliseconds the occluder has done a complete rotation (360 degrees). If each LED has a flash time of 2.5 milliseconds, in that time the occluder has moved 9 degrees relative to its nominal position. A total flash time for four LEDs is 10 milliseconds. For a shutter speed of 60/1000 seconds provides time for a full rotation of the occluder and four flashes, so as to generate four half images of the fundus which are combined to provide an image of the entire field of view in one shot. It will be appreciated that an image of the entire field of view of the fundus may be obtained from multiples of at least two half images, e.g., four or more half images, depending on parameters such as available light level, shutter speed, and occluder rotation speed.

Movement artifacts may be caused by relative motion of the camera and the eye during image capture. These are minor if light bundles are arranged to be substantially parallel between the aspheric lens and the eye. Movement then has minimal effect on the position of the fundus in the camera image, at most it may cause an uneven illumination or a limitation in the overall size of the image. The occluder is preferably optically black, to reduce reflections (e.g. a grayish occluder would reduce the contrast and leave a visible structure on the image generated by the image sensor).

As illustrated in FIG. 13, and FIGS. 16, 17 and 18, a conventional digital reflex (D-SLR) mirror camera is used. In this embodiment, for example, a Nikon™ D90 was chosen due to its high light sensitivity (ISO6400) and the possibility of making movies or video recordings. The imaging sensor is a CMOS chip with 4288×2848 pixels (12.2 Mega pixels) measuring 236 mm×15.8 mm.

A standard objective lens 344 on the camera will collimate light (focus at infinity) and an additional achromatic lens 330 is needed to collimate light from the intermediate focus plane at 8.7 mm in front of the aspherical lens 302. The LEDs 320 are placed between the achromatic lens and the intermediate focus plane at distance of 105 mm from the intermediate focus plane 304.

To minimize the size of the camera adapter body, the focal length should be kept at a minimum. In addition, the combination of a standard objective and the achromatic lens must provide the appropriate enlargement factor.

In this embodiment, the short side of the CMOS image sensor is 15.8 mm. The desired image size at the intermediate focus plane is about 25 mm in radius. With a minimal focal length for the achromatic lens of 120 mm the cameras objective lens should have a minimum focal length of 75 mm. For example, an appropriate off the shelf lens available meeting these requirements is an 85 mm f/1.8 NIKKOR™ digital camera objective from Nikon The achromatic lens must have a focal length of 135 mm for an image plane of 25 mm radius. The choice of achromatic lenses available on the market is limited. The most compatible lens has a focal length of 150 mm. For example, an achromatic lens from Linos™, f=150.Ø63 mm, has appropriate performance characteristics. The resulting image size on the short size of the CMOS sensor is about 27.8 mm at the intermediate focus plane.

It will be appreciated in examples provided in embodiments described in detail above, that the specific camera, lenses and other optical elements are provided by way of example only, using available components. Alternative components may be selected having similar parameters. For example, modeling shows that some chromatic aberrations may occur along the edge of the field, with separation of colours. Other lenses may alternatively be used, or customized lenses may be designed, to reduce aberrations if required. However in the interests of keeping costs down, off the shelf components may be preferred.

In the optical layout described above, the camera objective may be advantageously focused at about 4.4 m, for example, rather than at infinity. The achromatic lens 430 can then be mounted more or less in the appropriate position, so that the intermediate focus plane is relatively sharp. Then the focus of the camera objective 344 can still be adjusted for an optimal focus. This can be done using a live image displayed on the LCD display at the back of the camera with which one can also zoom in on part of the image. Adjusting the sharpness can be done quickly and easily checked. This adjustment will typically be required only once during set up of the system.

The resulting fundus imaging system is considerably lighter in weight than existing portable systems and has low power requirements. Advantageously, power demand from the small motor drive 354, 356 for rotation of the occluder is minimal, and power demand for illumination is reduced when the individual LEDs 320 are selectively illuminated or flashed only when required. For example, tests provided superior battery life, for example, using 4AA batteries, compared with existing commercial systems.

With the optical elements as shown in the FIGS. 12, 15-17, the fundus image is inverted on the camera view finder or viewing screen with respect to the subject. If required, inclusion of suitable additional optical elements, or preferably, electronic compensation, provides for inversion of the image.

Notably, systems and methods according to embodiments of the present invention described above provide for a very large field of view of the fundus, up to 120 to 130 degrees, with excellent image quality and reduced reflections. Image quality was assessed for clarity, resolution, field of view in degrees of fundus images, and absence of all or most reflections.

If a wide field of view is not required, reflections may be further reduced by changing or adjusting the optics to decrease the field of view, for example to around 50 degrees. In other embodiments the system may be switchable between narrow and wide field of view. For example, it may be beneficial to take an initial wide field of view to survey the fundus and then take one or more additional images with reduced field of view and higher quality, to image one or more particular areas of interest.

Adjustment may also be required to accommodate a range of refractive errors of the eye under examination. The distance on the optical axis between the intermediate focal plane and the surface of the aspheric lens is about 8.7 mm. Myopia or hyperopia will cause the fundus image to be out of focus on the CMOS imaging sensor at the nominal configuration. To correct this, it is preferable that position of the aspheric lens can be adjusted, to accommodate an expected range of refractive errors. The aspheric lens is preferably adjustable to provide focus to accommodate a range of optical parameters typically encountered in a population of subjects, e.g. −9 to +9 diopters. Using lenses such as those described above, the variation in distance of the aspheric lens for −8 to +8 diopters would be about −1.1 mm to +1.1 mm. The sharpness of the image will remain largely similar to that with emmetropia. For refractive errors above +8 diopters, the sharpness of the image at the edge of the field would be reduced.

The system is preferably compact, providing for a working distance for contact or non-contact imaging of an eye located about 0.5 to 2 cm from the input lens, and with a field of view up to 130 degrees, a system length of about 15 cm. Optionally polarization, or colour filters for selection of a particular illumination provides, e.g. for enhanced imaging of specific features, fluoroscopic imaging or fluorescein modality, may be provided.

The system described above was designed for non-contact use, i.e. using a working distance of ˜10 mm between the aspherical ophthalmic lens and the eye. For this reason it is preferable that the housing 370 near aspheric objective lens is arranged to be less bulky than that shown schematically in FIG. 16, to enable the lens to be placed close to the face. For some applications, (e.g. particularly imaging eyes of smaller animals) it may be preferable to use a contact lens and index matching gel or liquid.

In other variants, the camera adapter may include a connector or coupling to allow the adapter to be powered from the camera battery or an external power source. Similarly, control circuitry may provide for varying degrees of coupling or integration of the camera controls with the control system of the adapter. Control circuitry may also provide variable or selectable speed of rotation of the occluder and synchronization to different patterns of illumination.

While the illumination system 310 as shown in FIG. 17 shows four fixed LEDs 320 arranged at 90 degree intervals in a ring on an annular support 312, and a rotatable semicircular occluder 350 configured as shown in FIG. 14, to provide for selective and or sequential illumination of half of the fundus, it will also be appreciated that other arrangements of the illumination source or sources, and the occluder may provide for selective and/or sequential illumination of half of, or a sector comprising part of the fundus which may be larger or smaller than exactly one half of the fundus field of view, for example one third or two thirds of the fundus.

While selective illumination of a smaller sector of the fundus field of view (less than half) may further reduce reflections, reduced illumination may also require longer exposures, or multiple rotations and flashes. Instead of a semicircular occluder, it is envisaged that the occluder may alternatively comprise another form or shape such as shown in FIG. 13E, which occludes more or less than one half of the field, or an occluder such as in FIG. 13F wherein the sector is defined by a curved or non-linear boundary, if such an occluder provides acceptable image quality over the required field of view. If required, the occluder may be suitably shaped to leave a small central area open, i.e. to provide illumination in each flash to a central area of the fundus.

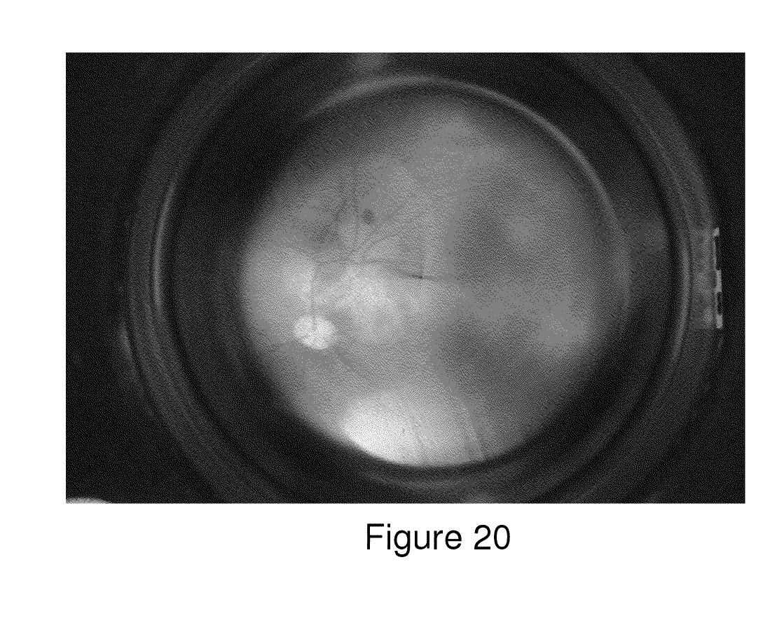

An illumination source comprising four LEDs and a rotatable occluder, as described above, provides sufficient illumination for four half images of good quality to be obtained and combined using a suitable rotation speed for the occluder and acceptably fast shutter speed. A sample photographic image of a pig eye is shown in FIG. 20. The illuminator may use alternative arrangements or configurations of LEDs to match corresponding occluder configurations. For example, an array of 2, 3 or more than four LEDS may be provided, which can be actuated (flashed) in an appropriate sequence, synchronized with rotation of the occluder and shutter operation. With fewer LEDs (less illumination), more rotations may be required to accumulate an image of sufficient quality. It is also envisaged that the illumination source may alternatively comprise other types of LEDs, or other solid state light sources, directly mounted on the ring illuminator, or alternatively a light source may be coupled via a fiber or light guide to provide the required arrangement of point light sources at appropriate positions for selective illumination of part, such a half, of the fundus to be imaged.

### Fundus Imaging Lens

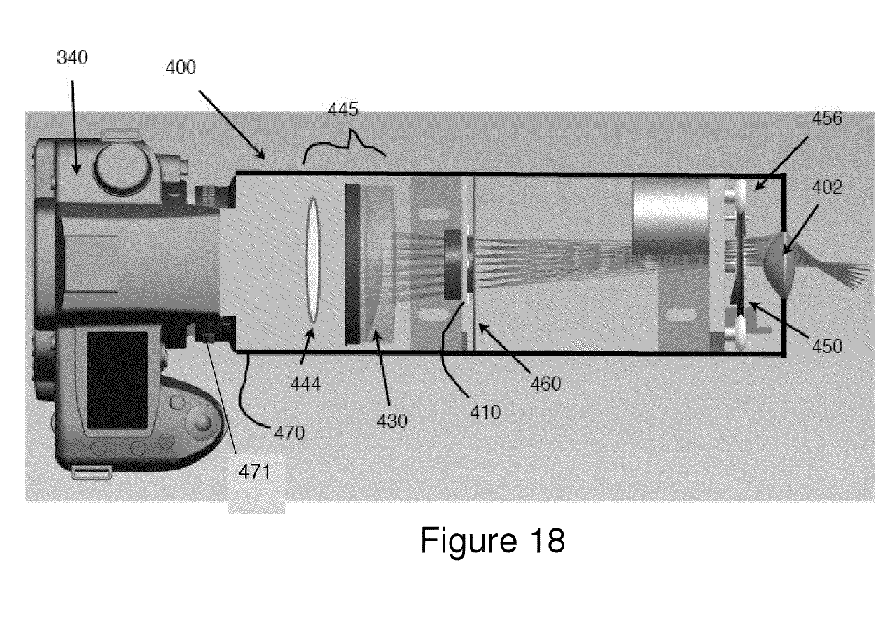

In another embodiment, shown in FIG. 18, the fundus imaging system 400 is provided as a conventional lens assembly for an interchangeable lens camera 340. In this case, the optical elements of the fundus imaging system are similar to those shown in FIG. 12 (and similarly numbered), but the second optical element 445 comprises one or more lenses 430, 444 that replace the functions of a separate camera objective and the achromatic coupling lens. The components would be supported in a standard lens housing 470, which would be mounted to the camera body 440 using a conventional lens mount 471, with associated power and control connections, as appropriate. As described above, conveniently, such an assembly may also comprise a handle with controls to facilitate hand held use.

In another form, a fundus imaging lens may comprise an assembly of a suitable camera objective lens with the fundus imaging adapter 300 described above.

### Fundus Imaging Camera

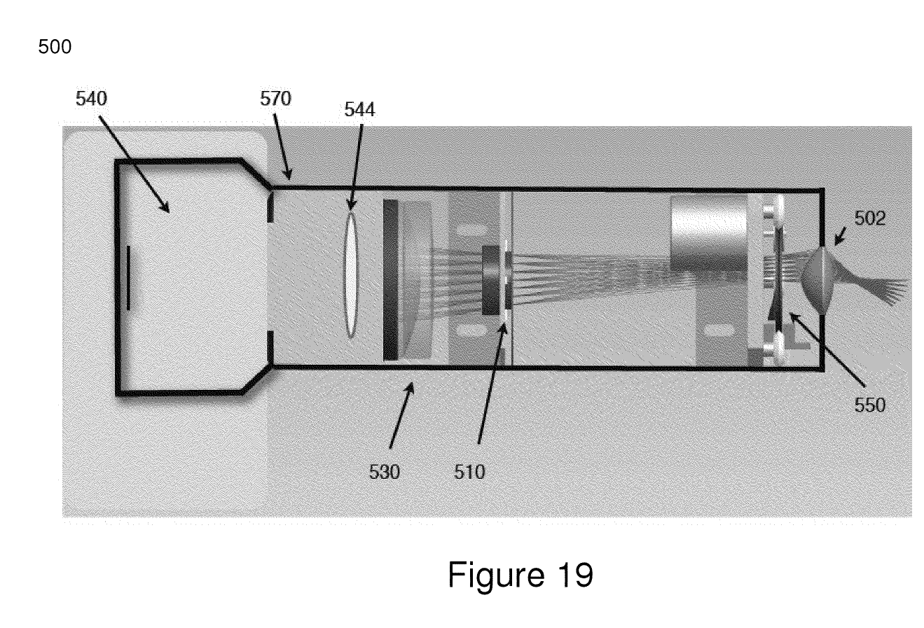

In yet another embodiment (FIG. 19) a fundus imaging system comprises a standalone fundus camera or imaging system 500, wherein the optical elements, which are similar to those shown in FIGS. 10 and 11, are customized and integrated within a housing 570 of the camera assembly comprising other optical elements of the camera, including the imaging sensor such as a CCD imaging device 542. Other elements are similar to those in other embodiments and are correspondingly numbered. A specialized fundus camera or retinal camera may thus be provided having a fixed lens system attached to the camera body 540.

Alternatively, a detachable or changeable lens module, which provides for fundus imaging such as shown in FIG. 18, may be used with a standard camera body.

Optionally, it is envisaged that camera systems as described above may be designed more specifically to provide desired features and functions for ophthalmic applications, e.g., appropriate imaging modes and control system, image processing, stabilization, colour filters or different colored light sources (LED or other) for specialized illumination, and/or to accommodate angiographic modalities in both the visible and infrared spectrum. For example, red illumination may be used for oxymetry, green illumination for red free images to eliminate blood and look at choroidal details, or infrared illumination to facilitate focusing without dilation, or to view other details of the fundus. For some applications, a camera comprising an image sensor with sensitivity for infrared imaging may be desirable. Thus, for example, instead of a set of four white light LEDs as shown in FIG. 13, a set of four groups each comprising a white light LED and separate sources for other types of illumination which may be actuated selectively or sequentially for different imaging modalities.

Alternatively, a fundus camera system may be provided which eliminates some non-essential features or functions of a regular camera, and includes only necessary elements and functions for operating the camera as a fundus imaging system, for example for a more convenient form factor, reduced cost or weight.

### Dual Occluder System

In the embodiments described above, a second rotating occluder element may be provided to substantially reduce back reflections from surfaces of the cornea. For example, in a system similar to that shown in FIGS. 12 to 17, a second semicircular occluder element may be provided closer to the plane of the LEDs or coplanar with the LEDs 320, and aligned with the first rotating occluder 350, to block light reflected from the corneal surface. On the other hand, a second rotating occluder itself gives rise to back reflections from the multiple light source elements 320, towards the camera aperture. Thus, in an alternative preferred embodiment comprising first and second occluder elements, beneficially a single light source is mounted on the rotatable second occluder element and rotated synchronously with the first occluder element. This arrangement has the benefit that the LED may remain on throughout rotation, and image data from multiple exposures of sequential sectors of the field of view of the fundus, as the illumination is rotated or scanned in around the entire field of view.

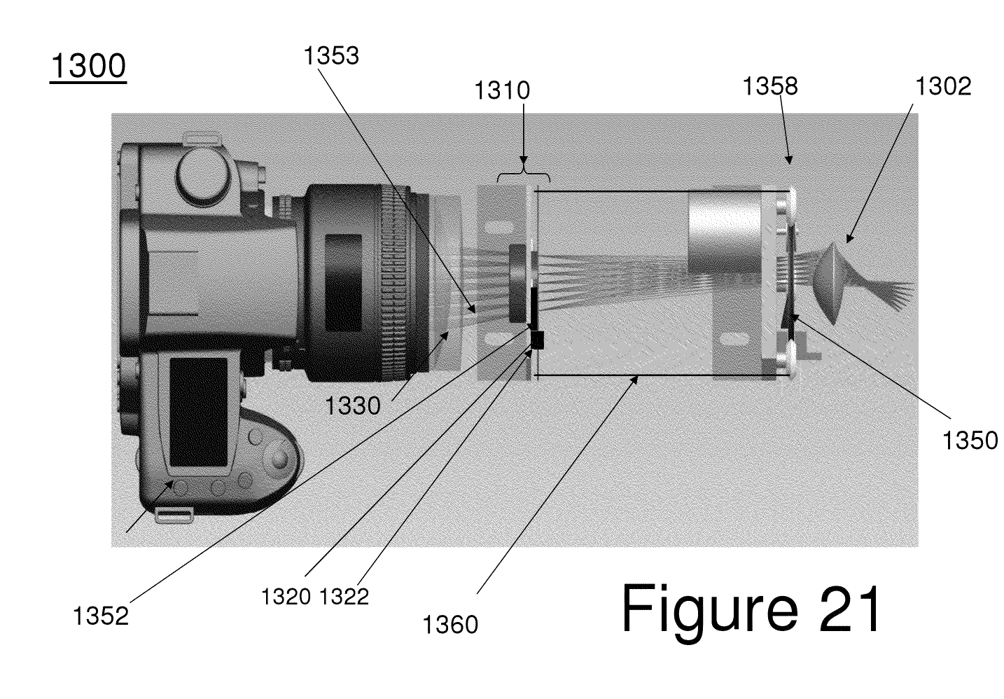

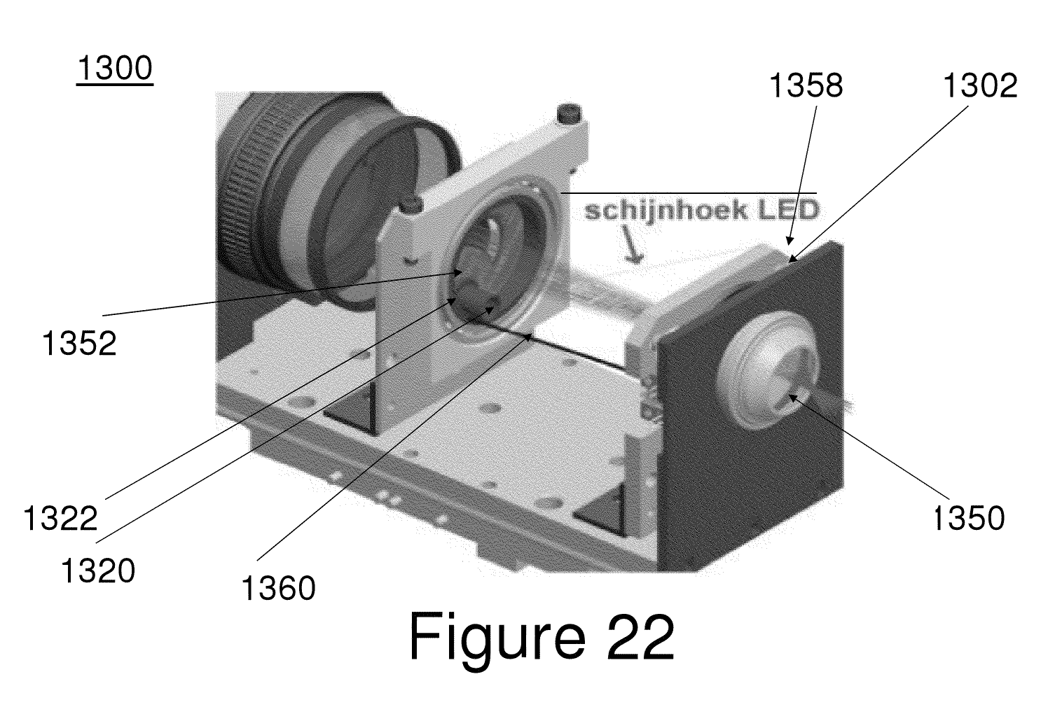

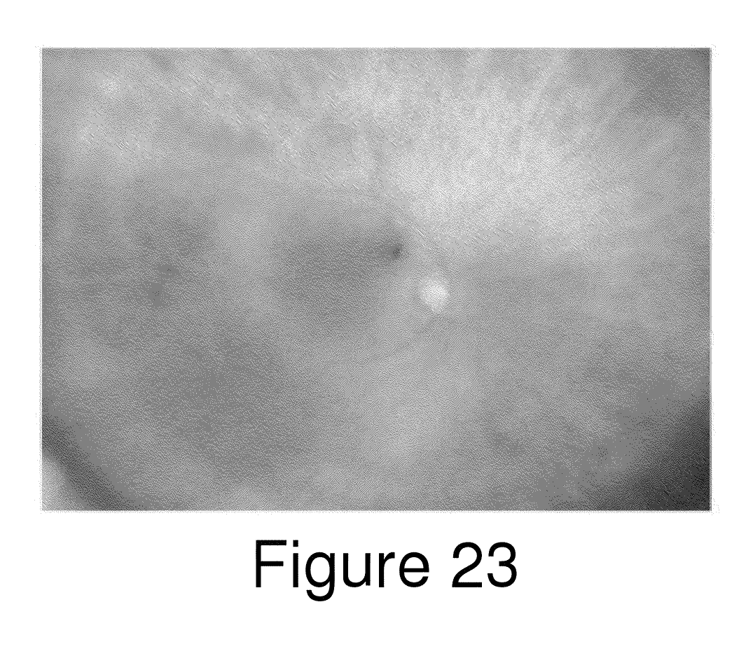

A fundus imaging system 1300, according to this alternative embodiment, is shown schematically in FIGS. 21 and 22. As illustrated in FIG. 21, this system 1300 is similar to that described above and shown in preceding FIGS. 12 to 17, and like elements are labeled with similar reference numerals incremented by 1000. However, the illuminator 1310 differs from the preceding embodiments. Instead of the plurality of LED light source elements 320, arranged concentrically in a ring that are operated in sequence to illuminate sequential sectors (FIG. 17), a light source element 1320 is radially spaced from the optical axis, and is rotatable around the optical axis (FIG. 21) together with first and second occluder elements 1350 and 1352 which are synchronously rotatable. As shown in FIGS. 21 and 22, the light source element 1320 is supported on a rotatable mounting. Advantageously, the light source element, comprising a single LED, or a closely spaced group of LEDs, is mounted on a second occluder, i.e. a semicircular occluder element 1352 which is aligned to the first occluder element 1350 and rotated synchronously therewith. The second occluder element 1352 may be coupled physically to the first occluder element 1350 so that synchronous rotation of the two occluder elements 1350, 1352 and the light source 1320 may be achieved with one motor drive 1358. The first and second occluders may be physically coupled, e.g. by a rod, rods or tubular coupling 1360, so that a single motor drive may be used to synchronously rotate both occluders 1350 and 1352 with light source element 1320. An element such as a baffle 1322, e.g. in the form of a small cylindrical tube, preferably surrounds the light source element 1320 to provide a more directed beam of illumination and reduce stray light which may give rise to spurious reflections. The light source element may, for example, comprise a compact group of LEDs, e.g. a white light LED, a IR LED for focusing and infrared imaging purposes, and one or more LEDs of specific colours (e.g. red, green, or blue) for additional imaging modalities, as mentioned above.

The second rotating occluder element 1352 serves to substantially reduce reflections from surfaces of the cornea from reaching the plane of the LED, and is preferably a similar semicircular occluder, which is aligned with the first rotating semicircular occluder 1350. Of course, when IR illumination is used, the first and second occluders must be optically black at IR wavelengths of the illumination, as well as visible wavelengths. Occluders which are opaque to visible light may transmit IR and necessitates an additional foil or coating to attenuate IR radiation.

With the additional occluder and rotating LED illumination imaging of the retina with reduced reflections from the cornea may be achieved, even without use of a polarizer. Moreover, because the image is summed from image data obtained by sequential exposure as the illumination of a sector comprising, e.g., half the fundus, is continuously rotated, improved image quality in the central portion of the field of view may be achieved. By comparison, where four half images are combined from four flashes or exposures at several, e.g. four, specific positions, as described in preceding embodiments, image artifacts, such as a small darker cross in the centre of the image, may be apparent due to edge or alignment effects at the intersections the four separate sectors or half images. On the other hand, in a photographic image of the entire field of view of the fundus (see FIG. 24) obtained from scanning illumination through 360 degrees rotation with continuous illumination and the dual occluder system such artefacts may be reduced.

Other variations of the embodiments described will be apparent, for example a different physical form of the housing, or a different physical arrangement of the components as a handle, control buttons or switches. The fundus imaging system may have capability to interface with or be used in conjunction with other features of commercially available cameras, such as different imaging modes (single image, multiple fast image capture or video); image processing and correction, colour balance adjustment, autofocus, and image stabilization, inter alia. In digital imaging systems, images may be captured and stored on a memory card or other local memory device. For telemedecine applications or field use, for example, wireless communications/networking capabilities may be provided for electronically transmitting images and associated data for remote analysis. The system may be coupled to a global positioning system, or other position location system, for ease of localization.

Since other known fundus cameras are dedicated to the photograph parts of the eye, they are unable or have limited capabilities in other photographic settings. In embodiments of the invention, high portability and a prolonged independence from a power source would also make it useful as a field device in zone of conflict or natural disasters. In these situations, a camera would not only be required to document intraocular abnormalities such as the sequellae of trauma, but also document external injuries to the eye or other parts of the body. Used in a field situation, an appropriate camera could be of simple construction, robust, and critical parts should be relatively easy to replace.

Thus, the above-described embodiments of the invention are intended to be examples only. Alterations, modifications and variations can be effected to the particular embodiments by those of skill in the art without departing from the scope of the invention, which is defined solely by the claims appended hereto.

### INDUSTRIAL APPLICABILITY

Embodiments of the present invention provide a method of selectively and/or sequentially illuminating sectors of the fundus for imaging with reduced reflections, and a corresponding compact optical system for a fundus camera, camera adapter or camera lens system for fundus imaging, which provide for a wide angle of view of the fundus with sufficient image quality for screening and diagnostics. A solid state LED illumination system with rotatable occluder provides advantages over conventional ring illumination to reduce reflections. A system may be provided which is lightweight and portable, is low power and easy to use, so as to enable mobile use for human or veterinary ophthalmology in the field, e.g., in remote areas, in zones of natural or manmade disasters for telemedicine, or bedside use. A camera adapter may be provided for a standard, off the shelf camera, with the potential to offer a low cost solution for ophthalmology application e.g. in third world areas.

Although embodiments of the invention have been described and illustrated in detail, it is to be clearly understood that the same is by way of illustration and example only and not to be taken by way of limitation, the scope of the present invention being limited only by the appended claims.

1. A fundus imaging system comprising:
first and second optical elements defining an optical axis for alignment to an eye to be imaged;
the first optical element comprising an objective lens for focusing an intermediate image of the fundus of the eye at an intermediate focal plane;
the second optical element for coupling the intermediate image for imaging the fundus in an image plane of an image sensor;
illumination means comprising an off-axis light source and an occluder for selectively illuminating a sector of the fundus within a field of view; and
control means for sequentially positioning the illumination means for illumination of a first sector of the field of view of the fundus, actuating the light source and capturing an image of the first sector, and then, positioning the illumination means for illumination of a second sector of the field of view of the fundus, actuating the light source and capturing an image of the second sector.

2. A fundus imaging system according to claim 1, wherein the control means comprises means for synchronizing rotation of the illumination means about the optical axis to sequentially illuminate first and second sectors of the field of view of the fundus during an image capture period of the camera.

3. A fundus imaging system according to claim 2, wherein
the light source comprises a light source element positioned on a ring radially spaced around the optical axis, and located relative to the intermediate focal plane of the ophthalmic objective lens for focusing an off-axis spot of light in a pupillary plane of the eye to be imaged;
the occluder comprises an occluder element located between the light source and the ophthalmic objective lens for occluding light from the light source element from part of the field of view; and
wherein the control means provides for positioning the occluder element and light source element and actuating the light source for selectively illuminating respective first and second sectors of the field of view of the fundus during the image capture period.

4. A fundus imaging system according to claim 3 further comprising a second occluder element aligned to the first occluder for blocking back reflections from a corneal surface of the eye.

5. A fundus imaging system according to claim 3, wherein
the light source comprises a light source element radially spaced from the optical axis and rotatable around the optical axis, the light source element located relative to the intermediate focal plane of the ophthalmic objective lens for focusing an off-axis spot of light in a pupillary plane of the eye to be imaged;
the occluder comprises an occluder element located substantially at the intermediate image plane of the aspheric lens and rotatable around the optical axis, and
the control means providing for synchronously rotating the light source element and the occluder element to illuminate a selected sector of the field of view of the fundus.

6. A fundus imaging system according to claim 5 further comprising a second occluder element for blocking back reflections from a corneal surface of the eye, the second occluder element supporting the light source element, and the second occluder element being rotatable about the optical axis synchronously with the first occluder element for selectively illuminating successive sectors of the field of view of the fundus during the image capture period.

7. A fundus imaging system according to claim 3, wherein the light source means comprises:
a plurality of light source elements arranged at positions around the ring, and wherein the occluder comprises an occluder element located substantially at the intermediate image plane of the aspheric lens; and
the control means provides means for rotating the occluder element and synchronously activating a respective one of the plurality of light source elements at a corresponding position around the ring to illuminate a corresponding selected sector of the field of view of the fundus.

8. A fundus imaging system according to claim 7, wherein the occluder element comprises a semicircular occluder element and the light source elements at first and second positions around the ring that are actuable for sequentially illuminating first and second sectors comprising first and second halves of the field of view of the fundus as the semicircular occluder is rotated, for forming a combined image of first and second halves of the field of view of the fundus during at least one full rotation of the occluder.

9. A fundus imaging system according to claim 7, wherein the occluder comprises a semicircular occluder and the plurality of light sources comprises four light sources arranged around the ring at 90 degree intervals and operable to flash in sequence synchronized to a speed of rotation of the occluder for sequentially illuminating a half of the fundus at 90 degree intervals.

10. A fundus imaging system according to claim 9, wherein the control means further comprises means for synchronous rotation of the occluder and actuation of the light source flashes to combine half images of the field of view of the fundus from at least one full rotation of the occluder during a single image capture period.

11. A fundus imaging system according to claim 3, wherein each light source element comprises a group of one or more LEDs, or one or more LEDs coupled to the a respective light source position via a light guide.

12. A fundus imaging system according to claim 9, wherein each light source element comprises a group of one or more LEDs, or one or more LEDs coupled to a respective light source position via a light guide.

13. A fundus imaging system according to claim 1, wherein the objective lens comprises an ophthalmic lens.

14. A fundus imaging system according to claim 1, wherein the objective lens comprises a double aspheric lens.

15. A fundus imaging system according to claim 1, wherein the objective lens comprises a wide angle ophthalmic lens for imaging up to 130 degrees of the field of view of the fundus.

16. A fundus imaging system according to claim 1, wherein the focus position of the objective lens is adjustable for refractive errors from +8 to −8 diopters.

17. A fundus imaging system according to claim 1 wherein the second optical element comprises an achromatic coupling lens.

18. A fundus imaging system according to claim 11 wherein the plurality of light source elements provide for imaging modalities using at least one of white light, red light, green light and infrared illumination.

19. A fundus imaging system according to claim 3, wherein the occluder element is oriented to direct reflections at substantially 10 degrees off-axis.

20. A fundus imaging system according to claim 1, further comprising a polarizer.

21. A fundus imaging system according to claim 1, further comprising means for recording data associated with a captured image comprising one or more of patient identification, other patient information, position, time and location.

22. A camera adapter comprising the fundus imaging system according to claim 1, wherein the second optical element comprises an achromatic lens for coupling the intermediate image through a camera objective lens for imaging on an image sensor of the camera.

23. A fundus imaging lens for a camera comprising the fundus imaging system of claim 1, mounted within a lens housing comprising a lens mount for coupling to a body of the camera and, wherein, the second optical element comprises a camera objective lens for coupling the intermediate image to the image sensor of the camera.

24. A method for fundus imaging comprising selectively illuminating a first sector of a field of view of the fundus, and capturing an image of the first sector, selectively illuminating a second sector of the same field of view of the fundus and capturing an image of the second sector, and combining images of first and second sectors.

25. A method according to claim 24, comprising sequentially illuminating sectors comprising one half of the field of view of the fundus, and combining two or more half images to obtain an image of the field of view of the fundus.

26. A method according to claim 24, wherein combining said images comprises sequentially illuminating at least first and second sectors within a single image capture period.

27. A method according to claim 24, for fundus imaging in a fundus imaging system having an optical axis defined by optical elements of the imaging system comprising an ophthalmic objective lens for optical alignment with an eye to be imaged, the method comprising:
providing a light source at a point position on a ring around the optical axis, and located relative to an intermediate focal plane of the ophthalmic objective lens for focusing an off-axis spot of light in a pupillary plane of the eye to be imaged;
from a first light source position selectively illuminating the first sector of a field of view of the fundus and capturing image data from the illuminated first sector of the fundus;
from a second light source position selectively illuminating the second sector of the field of view of the fundus and capturing image data from the illuminated second sector of the fundus; and
combining image data from at least first and second sectors within a single image capture period, to provide an image of at least part of the field of view of the fundus.

28. A method of illumination for a fundus imaging system according to claim 27, wherein selectively illuminating comprises positioning an occluder near an intermediate focus point of the aspheric lens to occlude light from the first light source position to other sectors during illumination of the first sector of the fundus; and rotating the occluder to occlude light from the second light source position to other sectors during illumination of the second sector the fundus.

29. A method of illumination for a fundus imaging system according to claim 28, comprising selectively illuminating the first sector comprising a first half of the fundus and capturing a first image, selectively illuminating the second sector comprising a second half of the fundus and capturing a second image, and combining the images of the first and second half of the field of view of fundus.

30. A method of illumination for a fundus imaging system according to claim 24, wherein images from illumination at least the first and second sectors of the fundus are recorded by synchronous rotation of an occluder and light source position around the optical axis and sequentially illuminating the first sector and the second sector of the fundus during a single shutter exposure period of the imaging system, to provide a combined image of the first and second sectors of the fundus.

31. A method of illumination for a fundus imaging system according to claim 29, comprising rotating an occluder and synchronously activating the light source to flash and sequentially illuminate successive halves of the fundus at 90 degree intervals of rotation during a single shutter exposure period of the imaging system, and capturing the combined images from successive illumination flashes obtained during at least one full rotation of the occluder.

32. An illumination system for wide angle imaging through an orifice, comprising:
first and second optical elements defining an optical axis for alignment to the orifice through which a subject is to be imaged;
the first optical element comprising an objective lens for focusing an intermediate image of the subject at an intermediate focal plane;
the second optical element for coupling the intermediate image for imaging the subject in an image plane of an image sensor;
illumination means comprising an off-axis light source positioned on a ring radially spaced around the optical axis, and located relative to the intermediate focal plane of the objective lens for focusing an off-axis spot of light in the plane of the aperture between the objective lens and the subject to be imaged, and a rotatable occluder located near an intermediate focal plane between the light source and the objective lens for occluding light from the light source from part of the field of view; and
control means for synchronizing, during an image capture period, sequential rotation of the occluder to a first position and actuation of the light source at a respective position for illuminating of a first sector of the field of view of the subject for capturing an image of the first sector, and then, rotation of the occluder to a second position and actuation of the light source at a respective position for illuminating a second sector of the field of view of the subject for capturing an image of the second sector, and combining images of first and second sectors of the subject.
